(19) United States
(12) Patent Application Publication
(10) Pub. No.: US 2009/0151401 A1
(43) Pub. Date: Jun. 18, 2009

(54) Restraint

(76) Inventor: Gustave C. Phanstiel, St. Petersburg, FL (US)

Correspondence Address: Gray Robinson;ATTN: STEFAN V. STEIN/ IP DEPT., 201 N. Franklin Street, Suite 2200, Post Office Box 3324, TAMPA, FL, 33601-3324, US

(21) Appl. No.: 11/958,458

(22) Filed: Dec. 18, 2007

(51) Int. Cl. A61F 5/37

(52) U.S. Cl. 70/16

(57) ABSTRACT

Disclosed is an improved restrain for immobilizing an individual. The restraint includes a central support that is secured in front of an individual and which simultaneously secures the neck and thighs. The restrain thereby forces the individual to bend at both the waist and the knees to severely limit the individual's range of movement. The restraint finds particular application in connection with law enforcement activities or other situations where it is necessary to easily and effectively immobilize an individual.

### BACKGROUND OF THE INVENTION

1. Field of the Invention

The present invention relates to an improved restraint. More particularly, the present invention relates to an improved restraint that immobilizes an individual via a support that is secured to the neck and thighs.

2. Description of the Background Art

The background art contains several examples of restraining devices. For example, U.S. Pat. No. 5,400,623 to Bota discloses an leg/ankle arrestor. The device is an elongated restrain that is secured behind the neck of the suspect being restrained. The upper end of the restrain is secured to a suspect's neck, while the foot of the suspect can be secured to the other end of the restraint. The suspect's hands can also be secured to the restraint. Likewise, U.S. Pat. No. 3,621,681 to Mikesic discloses a restraint that includes a handle that can be secured between handcuffs and leg encircling clips.

Although each of the above referenced inventions achieves its individual objective, they all suffer from a common problem. Namely, none of the referenced inventions immobilizes an individual by securing their neck and thighs. Nor do the referenced inventions require an individual to bend at the waist and knees to limit the individual's range of motion.

### SUMMARY OF THE INVENTION

It is therefore one of the objectives of this invention to provide an improved restraint to easily and effectively immobilizes an individual.

It is also an object of this invention to provide a restraint that includes a central support that is secured in front of the user.

Still another object of this invention is to provide a restraint that requires an individual to bend at the waist and knees.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a fuller understanding of the nature and objects of the invention, reference should be had to the following detailed description taken in connection with the accompanying drawings in which:

FIG. 1 is a front elevational view of an individual immobilized by the restrain of the present invention.

FIG. 2 is a side elevational view of an individual immobilized by the restrain of the present invention.

FIG. 3 is a front elevational view of the restraint of the present invention.

FIG. 4 is a detailed view taken along line 4-4 of FIG. 3.

FIG. 5 is a detailed view taken along line 5-5 of FIG. 3.

FIG. 6 is a detailed view taken along line 6-6 of FIG. 3.

FIG. 7 is a detailed view of one of the wrist collars of the present invention.

Similar reference characters refer to similar parts throughout the several views of the drawings.

### DETAILED DESCRIPTION OF THE PREFERRED EMBODIMENT

The present invention relates to an improved restraint for immobilizing an individual. The restraint includes a central support that is secured in front of an individual and which simultaneously secures the neck and thighs. The restrain thereby forces the individual to bend at both the waist and the knees to severely limit the individual's range of movement. The restraint finds particular application in connection with law enforcement activities or other situations where it is necessary to easily and effectively immobilize an individual.

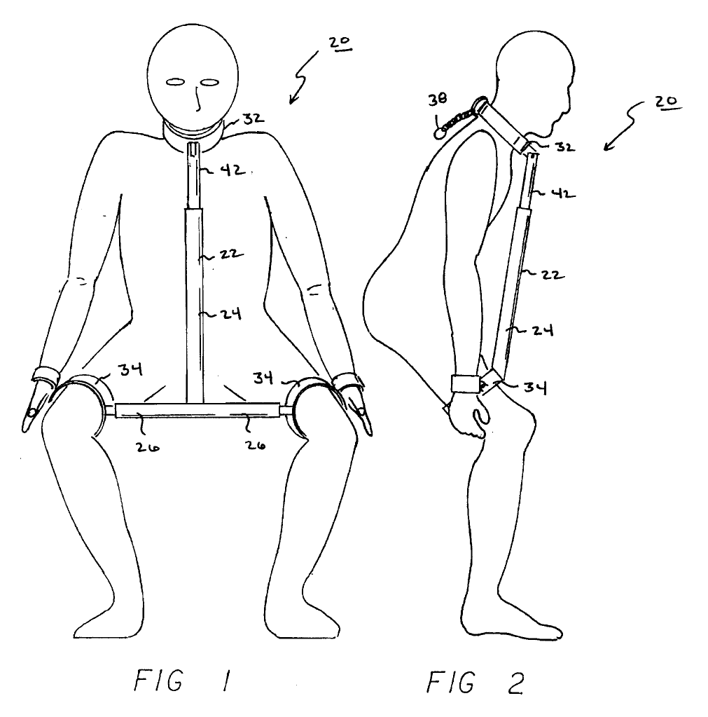

With reference now to FIGS. 1, 2 and 3, the device 20 along with its primary support 22 are depicted. This primary support 22 is preferably formed from aluminum. However, those skilled in the arts will appreciate that the primary support 22 can readily be formed from other suitable materials. As depicted, primary support 22 is in the form of an inverted “T” shape. More specifically, primary support 22 includes an upper central extent 24 and two lower lateral extents 26. Each of the three extents are hollow and share a similar construction. With particular reference to FIG. 3, the adjustment apertures 28 associated with each extent are depicted. Namely, a series of equally spaced apertures 28 are formed through a distal end of each of the three extents.

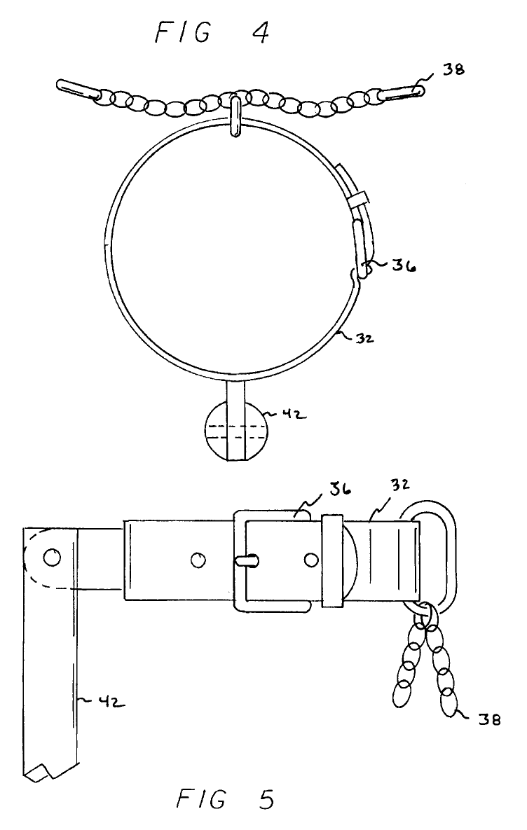

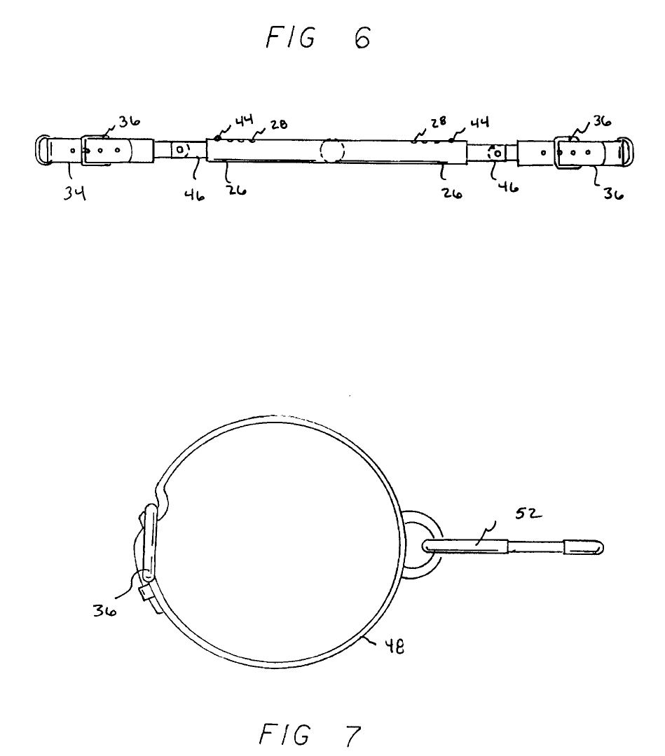

The three extents are adapted to be interconnected to a neck collar 32 and opposing thigh collars 34 in a manner described in greater detail hereinafter. FIGS. 4 and 5 are detailed views of the neck collar 32. As illustrated, neck collar 32 is formed from a belt with a buckle 36 positioned along its length for use in selectively adjusting the size of the collar 32. Buckle 36 thus permits collar 32 to be adjustably secured about an individual's neck. With continuing reference to FIGS. 4 and 5, a pair of hand restraints 48 are secured to neck collar 32 via chain 38. The function of these hand retrains 48 is described in greater detail hereinafter.

Neck collar 32 is secured to the central extent by way of a insert 42. More specifically, an aluminum insert 42 is pivotally secured to neck collar 32. A spring biased detent 44 is preferably formed along the length of insert 42. Thus, neck collar 32 can be removably secured to the primary support 22 by positioning insert 42 within the hollow central extent 24 of support 22. In this regard, the outer diameter of insert 42 is preferably slightly smaller than the inside diameter of the of central extent 24. Insert 42 is locked within central extent 24 by the spring biased detent 44 being received within one of the adjustment apertures 28. Furthermore, detent 44 can be received within any of the series of apertures 28 to thereby permit a telescopic adjustment between the insert 42 and central extent 24.

The pair of thigh collars 34 is illustrated in FIG. 3. Each of the thigh collars 34 shares a similar construction so only one will be described in detail. As with the neck collar 32, each thigh collar 34 is formed from a belt that includes a buckle 36 along its length for purpose of selectively adjusting the belt length 34. Each of the thigh collars 34 further includes an insert 46 that is pivotally secured thereto. Each insert 46 further includes a spring biased detent 44 formed along its length. As illustrated in FIG. 3, each insert 46 is inserted within one of the lateral extents 26 of the primary support 22 in a telescopic and adjustable manner. As with the neck collar 32, the detents 44 and adjustment apertures 28 allow the inserts 46 to be secured retained within the lateral extents 26 at any of number of different positions. This permits the restraint to be utilized by individuals of varying size.

With reference now to FIG. 7, the restraint of the present invention further includes wrist collars 48. Each wrist collar 48 is formed from a belt and includes a buckle 36 along its length for use in selectively adjusting the size of the belt 48. This permits the wrist collars 48 to be adjustably secured to a particular individual's wrist. Each of the wrist collars further includes a clip 52 for selectively securing the wrist collar 48 to one of the hand restraints 38 of the neck collar 32. Alternatively, the clips 52 can be secured to a restrained formed upon the thigh collars 34.

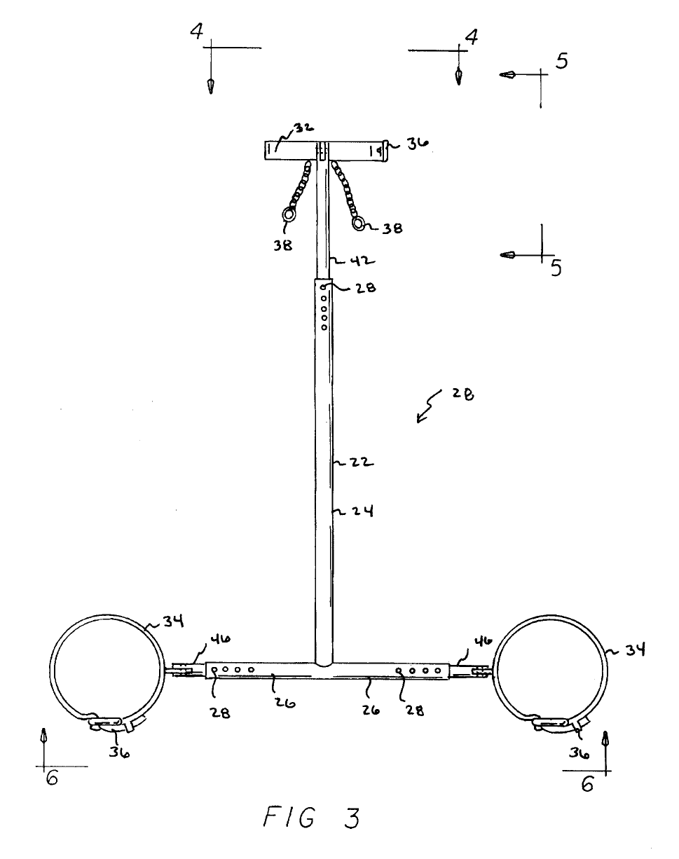

In use, and as noted in FIGS. 1 and 2, the neck collar 32 is secured to an individual's neck and the thigh collars 34 are secured to the individual's thighs. The collars are so positioned so that the central support 24 is positioned in front of the individual. The individual inserts 42 and 46 are then selectively positioned within the distal ends of the central and lateral extents 24 and 26. The insert of the neck collar 42 is preferably positioned within the central extent 24 so as to force the individual into a slight crouch. The individual is also forced to bend their knees by way of the thigh collars 34. All of this serves to effectively immobilize the individual. Finally, the hands of the user are restrained by attaching the wrist collars 48 to either the neck collar 32 or the thigh collars 34.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

Now that the invention has been described,

What is claimed is:

1. An improved restraint for immobilizing an individual comprising in combination:
a primary support formed from aluminum and having two lateral extents and one central extent, each of the extents being hollow and including a series of adjustment apertures adjacent a distal end thereof;
a neck collar formed from a belt, a buckle formed along the belt for selectively adjusting the size of the collar, a pair of hand restraints secured to the neck collar, an insert pivotally secured to the neck collar and having a spring biased detent formed thereon, the insert being inserted within the central extent of the primary support and being adjustably secured therein by way of the adjustment apertures and spring biased detent;
a pair of thigh collars, each thigh collar being formed from a belt and including a buckle formed along the belt for use in selectively adjusting the size of the belt, each of the thigh collars further including an insert pivotally secured thereto, with a spring biased detent formed on the insert, each insert being inserted within one of the lateral extents of the primary support and being adjustably secured therein by way of the adjustment apertures and spring biased detents;
a pair of wrist collars, each wrist collar being formed from a belt and including a buckle formed along the belt for use in selectively adjusting the size of the belt, each of the wrist collars including a clip for selectively securing the wrist collar to one of the hand restraints of the neck collar;
the restraint immobilizing an individual by securing the lateral extents of the primary support between the thighs of the individual with the individual thigh collars being secured to the thighs of the individual, the support being further secured by securing the neck collar to the neck of the individual such that the central extent of the support is positioned along the torso of the individual.

2. An improved restraint for immobilizing an individual comprising in combination:
a primary support having two lateral extents and one central extent, each of the extents being hollow;
a neck collar formed from a belt, an insert pivotally secured to the neck collar and being adjustably inserted within the central extent of the primary support;
a pair of thigh collars, each thigh collar being formed from a belt, each of the thigh collars further including an insert pivotally secured thereto with each insert being inserted within one of the lateral extents of the primary support.

3. The restraint as described in claim 2 wherein a spring biased detent is employed in retaining the inserts within the central and lateral extents.

4. The restraint as described in claim 2 wherein the belts of the neck and thigh collars are adjustable by way of a buckle.

5. The restraint as described in claim 2 further comprising a pair of wrist collars, each wrist collar being formed from a belt and including a buckle formed along the belt for use in selectively adjusting the size of the belt, each of the wrist collars including a clip for selectively securing the wrist collar to the neck collar.
